(19) United States
(12) Patent Application Publication
(10) Pub. No.: US 2015/0168366 A1
(43) Pub. Date: Jun. 18, 2015

(54) DIGESTION REACTOR AND ANALYTICAL DEVICE FOR DETERMINING A DIGESTION PARAMETER OF A LIQUID SAMPLE

(71) Applicant: Endress + Hauser Conducta Gesellschaft für Mess-und Regeltechnik mbH + Co. KG, Gerlingen (DE)

(72) Inventor: Marco Volker, Schwetzingen (DE)

(21) Appl. No.: 14/562,842

(22) Filed: Dec. 8, 2014

(30) Foreign Application Priority Data
Dec. 16, 2013 (DE) 10 2013 114 132.3

(51) Int. Cl. G01N 33/18; G01N 1/44

(52) U.S. Cl. CPC G01N 33/1806; G01N 1/44

(57) ABSTRACT

A digestion reactor for digesting a substance contained in a liquid sample, comprising: a digestion container for accommodating the liquid sample; and a heating apparatus comprising at least two heating shoes, especially heating shoes arranged opposite one another, each of which has a heating surface facing the digestion container and contacting a surface portion of the outer wall of the digestion container. An analytical device for determining a parameter of a liquid sample, especially a digestion parameter, such as chemical oxygen demand, total carbon content, total phosphate content, total iron content or total nitrogen content, can comprise such a digestion reactor as a well as a transport and dosing system, which is embodied to withdraw from a sample supply a predetermined volume of a liquid as a liquid sample and to transport the liquid sample into the digestion reactor; and a measuring transducer for registering a measured variable correlated, measured value of the liquid sample accommodated in the reactor.

The invention relates to a digestion reactor and to an analytical device for determining a digestion parameter of a liquid sample.

The determining of digestion parameters in liquid samples plays a role, for example, in process measurements technology or in industrial measurements technology, especially in the field of water and waste water treatment and/or in water and waste water analysis. Important examples of digestion parameters include chemical oxygen demand (COD), total carbon content and total nitrogen content (Nₜₒₜ).

Chemical oxygen demand is the oxygen equivalent amount of a chemical compound, usually a strong oxidizing agent, which is consumed by the oxidizable constituents contained in a certain volume of a liquid sample under the reaction conditions of a prescribed method. Serving as oxidizing agent, in such case, is frequently potassium dichromate. The COD value is, besides the total nitrogen content, an important parameter for classifying the degree of pollution in the case of river water and in waste water and clarification plants, especially those containing organic impurities.

In known methods for determining such digestion parameters, first of all, the liquid sample is mixed with a digestion agent in a digestion container, most often one embodied as a cuvette. This reaction mixture is heated, in given cases, under pressure, for a predetermined time in the digestion container. The substances to be detected, on whose concentration the given digestion parameter depends, are dissolved in such case by chemical reaction with the digestion agent. Depending on type of digestion parameter to be detected, either the consumption of the digestion means or a reaction with one or more additional reagents added to the reaction mixture brings about a change in the chemical and/or physical properties, e.g. the extinction, respectively absorption, of the liquid sample. This change can be detected, for example, by means of a suitable electrochemical transducer or, e.g. in the case of a change of extinction, respectively absorption, by means of a photometric, measuring transducer. The current value of the digestion parameter can be ascertained based on a measurement signal provided by the measuring transducer.

In the case of most methods for determining chemical oxygen demand, by way of example, a sample is treated with a known excess of an oxidizing agent and then the consumption of the oxidizing agent ascertained, for example, by back titration of the not consumed remainder. Photometrically ascertaining the consumption of oxidizing agent represents another option, e.g. when potassium dichromate serves as oxidizing agent. The amount of consumed oxidizing agent is converted into equivalent oxygen amount.

Known from the state of the art is a series of analytical devices for determining digestion parameters according to such methods. Described in German patent application DE 103 066 A1, for example, is an analytical device for photometrically determining the chemical oxygen demand of a liquid sample, wherein a cuvette-contained reaction mixture of the liquid sample and potassium dichromate as oxidizing agent is heated under pressure-tight closure for a digestion time at a temperature above the atmospheric boiling temperature of the reaction mixture. At the same time, the extinction of the reaction mixture in the cuvette as the digestion progresses is determined at least one fixed wavelength, wherein the change of the extinction serves as measure for the concentration change and therewith for the consumption of the oxidizing agent. Serving for heating the reaction mixture in the case of the analytical device known from DE 103 60 066 A1 is a heating apparatus, which is not specified in detail.

Known from Chinese patent application CN 102519780 A is an analytical device for determining the chemical oxygen demand of a liquid sample. The analytical device includes a glass digestion container for accommodating the liquid sample and the digestion means. A heating of the reaction mixture contained in the digestion container occurs by means of a heating wire, which is wound around the digestion container and carries an electrical current. This arrangement has various disadvantages. On the one hand, it is very complex to manufacture, because a number of protrusions from the outer wall of the glass digestion container must be provided for securement and guiding of the wire. This complicates the manufacture of the glass part. During the mounting of the heating wire, it must be heeded that such extends exactly in the guiding means, since otherwise short circuits could arise. In operation, the wire expands due to the heating during the digestion, so that it often no longer lies flushly against the digestion container. From this, the heat transfer to the glass sinks, while, simultaneously, the danger of short circuits increases.

It is, consequently, an object of the invention to provide a digestion reactor and an analytical device for determining a digestion parameter of a liquid sample, which overcome the described disadvantages.

The object is achieved by a digestion reactor as defined in claim 1 and by an analytical device having such a digestion reactor. Advantageous embodiments are set forth in the dependent claims.

The digestion reactor of the invention for digesting a substance contained in a liquid sample includes:
a digestion container for accommodating a reaction mixture containing the liquid sample; and
a heating apparatus comprising at least two heating shoes, especially heating shoes arranged opposite one another, each of which has a heating surface facing the digestion container and contacting a surface portion of the outer wall of the digestion container.

Two-dimensional heating shoes applied on the outer wall of the digestion container assure in comparison with a heating wire, which is wound around the digestion container and which has only a relatively low contact surface with the digestion container, a very much improved heat transfer and permit, thus, a faster heating of the reaction mixture. The danger of short-circuiting is likewise prevented by the application of heating shoes instead of a wire. The digestion reactor of the invention is thus significantly more reliable during operation than that known from CN 102519780 A. Guiding means in the outer wall of the digestion container required in the case of application of a heating wire can likewise be omitted in the case of the application of heating shoes. Thus, the manufacture of the digestion reactor is considerably simplified, compared with the reactor known from CN 102519780 A.

The digestion container can have a tubular section, wherein the heating surfaces of the heating shoes cover at least one third, preferably at least half, of the outer wall, especially preferably two thirds of the tubular section. In an embodiment, the digestion container can have a circularly cylindrical wall. The heating surfaces of the heating shoes can in this embodiment be concavely curved, wherein their radius of curvature is matched to the cross section of a cylinder defined by the outer wall of the digestion container in such a manner that the heating surfaces lie (lushly in contact with the outer wall.

If in this embodiment the two heating shoes are embodied identically, arranged opposite to one another and the heating surfaces have in sum a smaller surface area than the cylindrical lateral surface of the cylinder defined by the outer wall, especially less than two third, preferably less than half, of the area of the cylindrical lateral surface, oppositely lying sections of the cylindrical lateral surface remain free. These free sections can serve as a window for the entry, respectively exit, of a photometric measuring path for registering an extinction, respectively absorption, of a reaction mixture contained in the digestion reactor.

The heating shoes can each have at least one bore, in which a heating element, especially a power resistor, is embedded. The heating element can be embedded in the bore by means of heat conductive paste or heat conductive silicone.

In an embodiment, the heating shoes are formed of a metal, especially aluminum. Possible air gaps between the heating surfaces and the outer wall of the digestion container can optionally be eliminated by means of a heat conductive paste.

In a preferred embodiment, the digestion container is formed of an optically transparent material, especially glass. This is especially advantageous when measuring radiation from the visible spectral range, the near infrared or the near UV is serving for photometrically measuring a concentration of a substance in the digestion container.

The digestion container can have at least one liquid transport line and a pressure equalizing opening, wherein the liquid transport line and the pressure equalizing opening are each closable by means of a valve, so that digestion can be performed under pressure.

In an embodiment, the heating apparatus can comprise, connected with the heating shoes, a holder, which is embodied to press the heating shoes in the direction of a surface normal to the outer wall of the digestion container and against the outer wall of the digestion container. In this way, dimensional tolerances of the digestion container can be overcome. The heating shoes can be provided rear side with ribs, in order to prevent a full surface contact of the heating shoes with the holder and an associated, undesired heat removal to the holder.

An analytical device of the invention for determining a parameter of a liquid sample, especially a digestion parameter, such as chemical oxygen demand, total carbon content, total nitrogen content, total phosphate content or total iron content, includes:
a digestion reactor according to one of the above described embodiments,
a transport and dosing system, which is embodied to withdraw from a sample taking location a predetermined volume of a liquid as a liquid sample and to transport the liquid sample into the digestion reactor; and
a measuring transducer for registering a measured variable correlated, measured value of the liquid sample accommodated in the reactor and, in given cases, mixed with one or more reagents.

The measuring transducer can have a photometric sensor with a light source for irradiating the digestion container along a measuring path with measuring light, and with a light receiver for registering the intensity of the measuring light emitted from the light source after passing through the measuring path, wherein the measuring path enters the outer wall of the digestion container in a region not covered by the heating shoes and escapes from the outer wall of the digestion container in a region not covered by the heating shoes. In an embodiment of the digestion container, in the case of which the outer wall has a cylindrical shape, and in the case of which the two oppositely lying heating surfaces have in sum a smaller surface area than the surface area of the cylindrical lateral surface formed by the outer wall of the digestion container, two oppositely lying sections of the outer wall, through which the measuring path extends, can remain free.

The analytical device can further comprise a number of liquid containers, which contain reagents to be added to the liquid sample for forming a reaction mixture, a standard solution and/or rinsing/washing liquid, and which are connected with the digestion container, wherein the transport and dosing system is embodied to withdraw from the liquid containers, in each case, a predetermined liquid amount and to transport such into the digestion container.

The transport and dosing system can have at least one pump and a metering container connected with the pump, wherein the liquid containers are connected via the metering container with the digestion container. The liquids to be dosed are in this embodiment transported by means of the pump, first of all, into the metering container, which serves for measuring a predetermined liquid volume.

The liquid containers can be connected with the metering container via liquid transport lines each of which is controllable by at least one valve, wherein the transport and dosing system has a central valve control mechanism, which is embodied to actuate at least a portion of the valves, especially all valves. The central control mechanism associated with one or more valves can be embodied, for example, as described in German patent application DE 102011075762 A1.

The analytical device can further comprise an evaluation and control system, which is embodied, especially by controlling the supply, and dosing, system, to guide the liquid sample and/or predetermined amounts of the liquids from the liquid containers into the digestion container and/or which is embodied, based on a measurement signal of the measuring transducer, to ascertain the parameter of the liquid sample. The evaluation and control system can comprise a data processing system, which includes at least one processor and a program memory, in which is stored a computer program, which can be executed by the processor and which serves for control of the analytical device and evaluation of the measurement signals produced by means of the measuring transducer for determining a value of the digestion parameter. For interaction with the analytical device, the evaluation and control system can include input means, e.g. a keyboard or one or more switches, and a display.

The invention will now be explained in greater detail based on the example of an embodiment illustrated in the appended drawing, the figures of which show as follows:

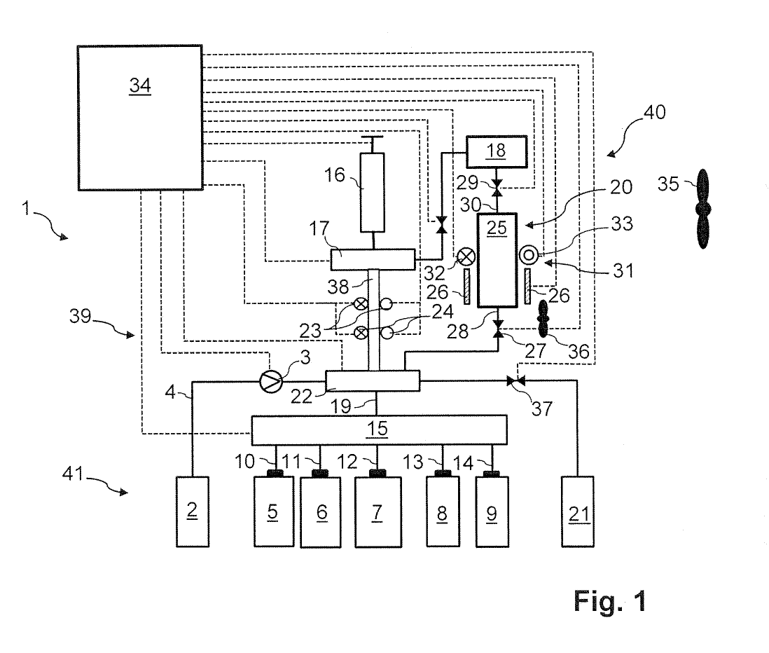

FIG. 1 a schematic representation of an analytical device for determining the chemical oxygen demand of a liquid sample;

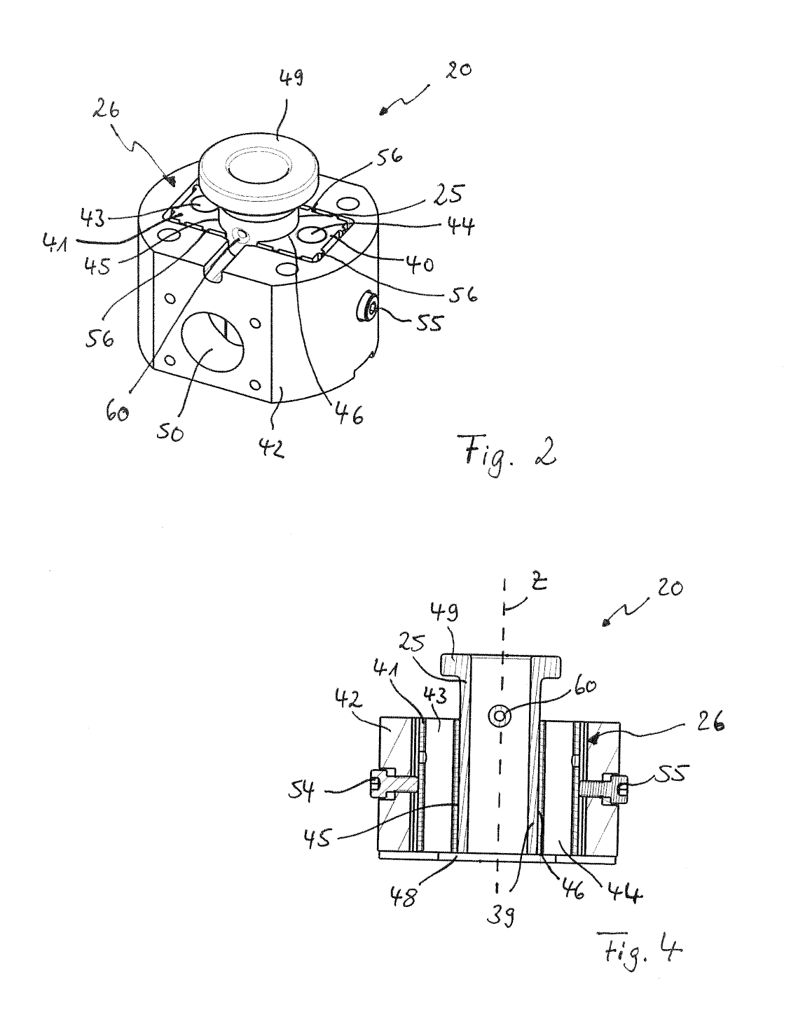

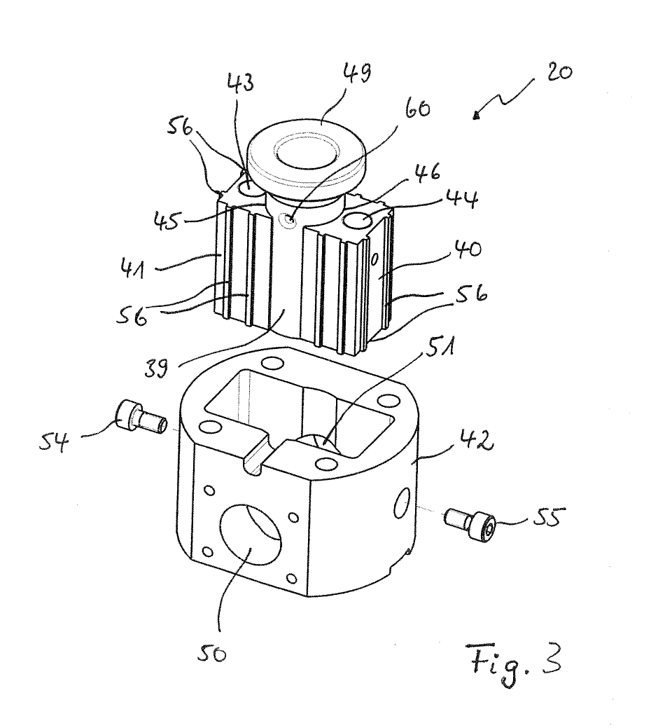

FIG. 2 a schematic representation of the digestion reactor of the analytical device illustrated in FIG. 1.

FIG. 3 a schematic, exploded view of the digestion reactor illustrated in FIG. 2; and

FIG. 4 a schematic cross section of the digestion reactor illustrated in FIGS. 2 and 3.

The analytical device 1 schematically shown in FIG. 1 serves for determining the chemical oxygen demand of a liquid sample of a monitored liquid present at a sample taking location 2. Although the example described here and in the following relates to an embodiment of an analytical device for determining the chemical oxygen demand, the invention is nevertheless equally applicable to analytical devices for directly determining the most varied of digestion parameters, for example, total carbon, total nitrogen, total phosphate or total iron.

The sample taking location 2 can be, for example, an open vat or flume or a closed container, for example, a pipeline. The liquid can be, for example, waste water to be treated in a clarification plant. Serving for removal of the liquid sample from the sample taking location 2 in the example shown here is a sample taking apparatus 3, which can comprise, for example, a pump. The liquid transport line 4 is connected via a connection 22 with a metering container 38. The metering container 38 includes two light barriers 23, 24, which serve for determining the fill level of a liquid in the metering container 38.

The analytical device 1 includes a number of liquid containers 5, 6, 7, 8 and 9, which contain reagents to be added to the liquid sample for determining the COD, and standard solutions for calibrating and/or adjusting the analytical device 1. In the example shown here, a first liquid container 5 contains an aqueous potassium dichromate solution as digestion agent, a second liquid container 6 contains an aqueous mercury sulfate solution for masking chloride ions contained, in given cases, in the liquid, and a third liquid container 7 contains sulfuric acid. A fourth liquid container 8 contains a first standard solution, which has a first predetermined chemical oxygen demand. A fifth liquid container 9 contains a second standard solution with a second chemical oxygen demand different from the first chemical oxygen demand. In the present example, the second standard solution is deionized water to provide a zero standard.

The liquid containers 5, 6, 7, 8, 9 are connected via liquid transport lines 10, 11, 12, 13, 14 with a liquid transport line 19 opening via the connection 22 into the metering container 38. The liquid transport lines 10, 11, 12, 13 and are each controlled by valve, wherein a here only schematically indicated, central valve control mechanism 15 serves for actuation of valves. Metering container 38 is connected with a piston pump 16, which is actuatable by means of a linear motor (not shown in FIG. 1). The metering container 38 is connected via the connection 17 and a valve with the atmosphere 18. By means of the valve, the metering container 38 and/or the piston pump 16 can be selectively connected with the atmosphere 18. The metering container 38 is connected, moreover, via the connection 22 with a digestion reactor 20, which simultaneously serves for the digestion of a liquid sample and as measuring cell for determining the chemical oxygen demand. The metering container is connected via the connection 22, moreover, with a liquid transport line leading to a waste container 21 and controlled by the valve 37.

The digestion reactor 20 comprises, formed of a transparent material, e.g. glass, a digestion container 25, which is heatable by a heating apparatus 26. Opening into the digestion container 25 is a liquid transport line 28 connectable by means of a first valve 27 selectively with the metering container 38 or with the waste container 21. Moreover, digestion container 25 includes, controlled by a second valve 29, a pressure equalizing line 30, by means of which the digestion container 25 is connectable with the atmosphere 18.

The sample taking apparatus 3, the metering container 38, the piston pump 16, the central valve control mechanism 15, the valves actuatable by the central valve control mechanism, as well as the valves 27, 29 form a transport and dosing system of the analytical device 1. The transport and dosing system serves for transport and dosing of the liquid sample, as well as of reagents to be added to the liquid sample, into the digestion reactor 20.

The analytical device 1 includes for ascertaining a measured value representing the chemical oxygen demand of the liquid sample a photometric sensor 31, which has a light source 32 and a light receiver 33. The light source 32 can comprise, for example, one or more LEDs, especially LEDs emitting light of different wavelengths, or one or more multi-LEDs, while the light receiver 33 can have one or more photodiodes. Measuring light emitted by the light source 32 irradiates the digestion container 25 along a measuring path extending through the reaction mixture contained in the digestion container 25 and then strikes the light receiver 33.

The photometric sensor 31 produces, as a function of the intensity of the light striking the light receiver 33, an electrical measurement signal, which, in given cases, is amplified and/or digitized by a sensor circuit (not shown). The light intensity striking the light receiver 33 depends on the extinction, respectively absorption, of the reaction mixture contained in the digestion container 25. The light source 32 in the present example is embodied in such a manner that it emits, as a measuring light, light of least one wavelength, whose absorption or extinction is a measure for the consumption of the digestion means serving for oxidation of oxidizable components of the liquid sample. In the present example, the digestion means is potassium dichromate. Thus, the electrical measurement signal produced by the photometric sensor 31 is a measure for the chemical oxygen demand of the liquid sample.

The analytical device 1 includes, finally, an evaluation and control system 34. This includes an electronic data processing system, which has one or more processors and one or more data and program memories. The evaluation and control system 34 is connected with the photometric sensor 31 and obtains from such the measurement signal, which is, in given cases, digitized and amplified. Stored in a memory of the evaluation and control system 34 is a computer program executable by the one or more processors and serving for ascertaining the chemical oxygen demand based on the measurement signal representing an extinction or absorption by the reaction mixture.

The evaluation and control system 34 is, moreover, connected with the individual components of the transport and dosing system of the analytical device 1, especially the pumps, the central valve control mechanism 15 and the individual valves 17, 22, 27, 29, in order to control transport of predetermined liquid amounts from the sample taking location and predetermined reagent amounts from the liquid containers 5, 6 and 7 into the digestion container 25 for performing a determining of the chemical oxygen demand. Equally, the evaluation and control system 34 can control the performing of calibration measurements by withdrawing by means of the transport and dosing system, instead of from the sample taking location 2, from one or both of the standard-liquid containers 8, 9 a predetermined amount of a standard solution as liquid sample. The evaluation and control system 34 can be embodied, based on such a calibration measurement, to conduct an adjusting of the analytical device 1. Moreover, the evaluation and control system 34 can be connected with the heating apparatus 26, in order to control the heating of a reaction mixture contained in the digestion container 25.

The analytical device can have a housing (not shown in FIG. 1) enclosing the liquid containers, the dosing, metering and supply system, the pressure reactor and the evaluation and control system. Arranged within the housing can be one or more housing ventilators 35. An additional, reactor ventilator 36 can be located in the direct vicinity of the digestion reactor 20.

FIGS. 2 to 4 show the digestion reactor 20 in more detail. FIG. 2 shows the vertically standing digestion reactor 20, FIG. 3 shows an exploded view of the digestion reactor 20 and FIG. 4 shows a cross sectional view of the digestion reactor 20.

The digestion container 25 is embodied as a vertically standing hollow cylinder of glass with a cylinder axis Z. The hollow cylinder includes a cavity 60, in which, when required, a temperature sensor can be arranged for registering a temperature measured value correlating with the temperature of a liquid sample contained in the digestion container 25. The lower region of the hollow cylinder ends in a first connection 48, to which a flange is adapatable surrounding a connection for liquid transport line 28 (not shown). The pressure equalizing line 30 is adapatable to a flange 49 arranged on the oppositely lying end of the hollow cylinder. The outer wall 39 of the digestion container includes besides the oppositely lying base surfaces of the digestion container comprising the line ends, a cylindrical lateral surface extending coaxially with the cylinder axis Z.

The heating apparatus 26 includes two oppositely lying, identically embodied, heating shoes 40, 41, which have concave heating surfaces 45, 46 applied against the outer wall 39 of the digestion container 25. The surface area of the heating surfaces 45, 46 is in sum smaller than the surface area of the cylindrical lateral surface of the outer wall 39. The thereby remaining free regions of the outer wall 39 can be traversed by the measuring path of the photometric sensor 31.

The heating shoes 40, 41 are in the present example formed of aluminum. Each has, extending parallel to the cylinder axis Z, a bore 43, 44, in which a power resistor is embedded as heating element. Used for embedding the power resistors can be, for example, heat conductive paste or heat conductive silicone. The heating shoes 40, 41 are seated in a holder 42, which has two oppositely lying, traversing openings 52, 53, in which screws 54, 55 are arranged acting on the respective rear sides of the heating shoes 40, 41. By means of the screws, the heating shoes 40, 41 are shiftable along a surface normal of the cylindrical lateral surface formed by the outer wall 39 and intersecting with the cylinder axis Z orthogonally, in order to press the heating surfaces 45, 46 against the outer wall 39 of the digestion container 25. In this way, dimensional tolerances of the digestion container 25 can be overcome. The holder includes, moreover, in the example shown here, two oppositely lying openings 50, 51, which serve to accommodate the light emitter 32 and the light receiver 33. The optical measuring path extending between light emitter 32 and light receiver extends preferably perpendicularly to the surface normal, along which the heating surfaces are caused to bear against the outer wall 39 of the digestion container 2.

The heating shoes 40, 41 have ribs 56 on their side surfaces not adjoining the outer wall 39 of the digestion container 25. Ribs 56 are surrounded by the holder 42 and extend axially parallel to the cylinder axis Z. Ribs 56 prevent full surface contact with the holder 42 and therewith undesired heat removal from the holder 42.

Process flow for photometrically determining the chemical oxygen demand of a liquid sample by means of the analytical device 1 is, for example, as follows:

First, by means of the sample taking apparatus 3, liquid is transported via the liquid transport line 4 from the sample taking location 2 into the metering container 38. In such case, the liquid transport lines leading to the liquid containers 5, 6, 7, 8, 9, 21 and the digestion reactor 20 are closed by valves. The metering container 38 is connected with the atmosphere 18 during the liquid transport via the liquid transport line 4 in the here described example. Alternatively, the piston pump 16 and the metering container can during the transport of the liquid via the liquid transport line 4 into the metering container 38 be isolated from the atmosphere 18, so that the piston pump 16 can support the liquid transport. The evaluation and control system 34 controls the metering of the liquid into the metering container 38 by means of the light barriers 23, 24. If in the metering container 38 a predetermined fill level is achieved, the evaluation and control system 34 ends the transport of liquid into the metering container 38. Thereafter, the metering container 38 is connected with the digestion container 25 by opening the valve 27 for opening the liquid transport line 28 opening into the digestion container 25. At the same time, the piston pump 16 and the metering container 38 are isolated from the atmosphere 18, to the extent that they were not already isolated from the atmosphere 18 in the case of transporting the liquid into the metering container 38. By means of the piston pump 16, the liquid contained in the metering container 38 is transported into the digestion container 25, wherein the light barriers 23, 24 can, in given cases, serve for fine metering.

Alternatively to the liquid sample removed from the sample taking location 2, for the case, in which a calibrating and/or adjusting is to be performed, in analogous manner, also a predetermined amount of the two standard solutions or a mixture of the two standard solutions can be transported from the containers 8, 9 into the digestion container 25. The further handling of the calibration measurement is identical to the following described additional steps of the COD determination.

To the liquid sample provided in the digestion container is added, respectively, a predetermined amount of sulfuric acid, mercury sulfate serving as masking means, as well as potassium dichromate serving as digestion agent. These reagents are dosed by means of the piston pump 16 with the cooperation of the central valve control mechanism 15, the valve 27 controlling the liquid transport line 28 of the digestion reactor 20 and the pressure equalizing valve 29 and are transported into the digestion container 25. The light barriers 23, 24 can serve again for metering.

Then, the liquid transport line 28 and the pressure equalizing line 30 opening into the digestion container 25 are closed by means of the valves 27 and 29 and the heating apparatus 26 turned on. The heating apparatus 26 heats the reaction mixture comprising the liquid sample and the added reagents located in the digestion container 25 to a temperature of about 175° C. at a pressure of 5 to 10 bar and holds this temperature constant. Upon the beginning of the heating procedure, virtually continuously, the extinction and/or absorption of the reaction mixture is determined by means of the photometric sensor 31. The extinction, respectively absorption, values are evaluated by means of the evaluating and control system 34.

As soon as a specified state is reached with reference to the extinction or absorption, the extinction or absorption measuring is ended. The specified state can be a minimum rate of change of the extinction or absorption, for example, a change of the extinction of less than one percent in 10 seconds. The measured value of the extinction or absorption present upon reaching the specified state is used by the evaluation, and control system for determining the chemical oxygen demand of the sample. After reaching the specified state, the digestion of the liquid sample can be ended and the heating apparatus 26 turned off. By means of the valves 27 and 37, the liquid transport line 28 of the digestion container 25 can be connected with the waste container 21 and used reaction mixture can be removed from the digestion container 25 via the liquid transport line 28 and transported into the waste container 21. For assuring a complete emptying of the digestion container 25, it is possible, first of all, only to open the valve 27 and to transport used reaction mixture by means of the piston pump 16, first of all, into the metering container 38, then to close valve 27, while simultaneously opening the valve 37 and transporting used reaction mixture from the metering container into the waste container 21. During this, the valves actuatable by the central valve control mechanism 15 and serving for opening and closing the liquid transport lines 10, 11, 12, 13 and 14, are closed, in order to prevent used reaction mixture from getting into the liquid containers 5, 6, 7, 8 and 9.

In an alternative method, the digestion of the liquid sample can be performed by heating the reaction mixture under pressure for a predetermined time, e.g. at 175° C. and a pressure of 5 to 10 bar for a time period of 30 to 120 minutes. A virtually continuous monitoring of the extinction, respectively absorption, is, in this case, not required. It can be performed, however, for example, for obtaining additional information. After expiration of the predetermined period of time, the extinction and/or absorption of the reaction mixture can be registered and used for determining the COD value of the liquid sample. The used reaction mixture can, such as above described, be cooled off and fed to the waste container 21.

All of the above described method steps are performed automatically under the direction of the evaluation and control system 34 in the here described example.

1-13. (canceled)

14. A digestion reactor for digesting a substance contained in a liquid sample, comprising:
a digestion container for accommodating the liquid sample; and
a heating apparatus comprising at least two heating shoes, especially heating shoes arranged opposite one another, each of which has a heating surface facing said digestion container and contacting a surface portion of the outer wall of said digestion container.

15. The digestion reactor as claimed in claim 14, wherein:
said digestion container has a tubular section; and
said heating surfaces cover at least one third, preferably at least half, of said outer wall of the tubular section.

16. The digestion reactor as claimed in claim 14, wherein:
each heating shoe has at least one bore, in which a heating element is embedded, especially by means of heat conductive paste or heat conductive silicone.

17. The digestion reactor as claimed in claim 14, wherein:
said heating shoes are formed of a metal, especially aluminum.

18. The digestion reactor as claimed in claim 14, wherein:
said digestion container is formed of an optically transparent material, especially glass.

19. The digestion reactor as claimed in claim 14, wherein:
said digestion container has at least one liquid transport line and a pressure equalizing opening;
said liquid transport line and said pressure equalizing opening are each closable by means of a valve.

20. The digestion reactor as claimed in claim 14, wherein:
said heating apparatus comprises, connected with said heating shoes, a holder, which is embodied to press said heating shoes in the direction of a surface normal to said outer wall of said digestion container and against said outer wall of said digestion container.

21. The analytical device for determining a parameter of a liquid sample, especially a digestion parameter, such as chemical oxygen demand, total carbon content, total phosphate content, total iron content or total nitrogen content, comprising:
a digestion reactor for digesting a substance contained in a liquid sample, comprising: a digestion container for accommodating the liquid sample; and a heating apparatus comprising at least two heating shoes, especially heating shoes arranged opposite one another, each of which has a heating surface facing said digestion container and contacting a surface portion of the outer wall of said digestion container;
a transport and dosing system, which is embodied, to withdraw from a sample taking location a predetermined volume of a liquid as a liquid sample and to transport the liquid sample into said digestion reactor; and
a measuring transducer for registering a measured variable correlated, measured value of the liquid sample accommodated in said digestion reactor and, in given cases, mixed with one or more reagents.

22. The analytical device as claimed in claim 21, wherein:
said measuring transducer has a photometric sensor with a light source for irradiating said digestion container along a measuring path with measuring light and with a light receiver for registering the intensity of the measuring light emitted from the light source after passing through the measuring path;
said measuring path enters an outer wall of said digestion container in a region not covered by heating shoes and escapes from the outer wall of said digestion container in a region not covered by the heating shoes.

23. The analytical device as claimed in claim 21, further comprising:
a number of liquid containers, which contain the reagents to be added to the liquid sample, a standard solution and/or rinsing/washing liquid, and which are connected with said digestion container, wherein:
said transport and dosing system is embodied to withdraw from said liquid containers, in each case, a predetermined liquid amount and to transport such into said digestion container.

24. The analytical device as claimed in claim 23, wherein:
said transport and dosing system has at least one pump and a metering container connected with said pump; and
said liquid containers are connected via said metering container with said digestion container.

25. The analytical device as claimed in claim 24, wherein:
said liquid containers are connected with said metering container via liquid transport lines, each of which is controllable by at least one valve; and
said transport and dosing system has a central valve control mechanism, which is embodied to actuate at least a portion of said valves, especially all valves.

26. The analytical device as claimed in claim 23, further comprising:
an evaluation and control system, which is embodied, especially by controlling said supply, and dosing, system, to guide the liquid sample and/or predetermined amounts of the liquids from said liquid containers into said digestion container and/or which is embodied, based on a measurement signal of said measuring transducer, to ascertain the parameter of the liquid sample.
